(19) United States
(12) Patent Application Publication
(10) Pub. No.: US 2025/0134926 A1
(43) Pub. Date: May 1, 2025

(54) COMPOSITION FOR ANTI-CANCER TREATMENT COMPRISING IMMUNOGENIC TUMOR-DERIVED EXTRACELLULAR VESICLES USING AN ENDOPLASMIC RETICULUM STRESS INDUCER AND METHOD FOR PRODUCING THE SAME

(71) Applicant: Research & Business Foundation SUNGKYUNKWAN UNIVERSITY, Suwon-si (KR)

(72) Inventors: Jae Hyung PARK, Suwon-si (KR); Chan Ho KIM, Suwon-si (KR); Kyung Hee HAN, Seoul (KR); So Hee KIM, Suwon-si (KR)

(73) Assignee: Research & Business Foundation SUNGKYUNKWAN UNIVERSITY, Suwon-si (KR)

(21) Appl. No.: 18/909,381

(22) Filed: Oct. 8, 2024

(30) Foreign Application Priority Data
Oct. 30, 2023 (KR) 10-2023-0146485

(51) Int. Cl. A61K 35/13; A61K 31/365; A61K 31/7004; A61K 31/7072; A61P 35/00; C12N 5/09

(52) U.S. Cl. CPC A61K 35/13; A61K 31/365; A61K 31/7004; A61K 31/7072; A61P 35/00; C12N 5/0693

(57) ABSTRACT

The present invention relates to a composition for anti-cancer treatment, the composition including immunogenic tumor-derived extracellular vesicles using an endoplasmic reticulum stress inducer, and a method for producing the same. The composition according to the present invention includes extracellular vesicles in which tumor immune-activating protein is increased and immune-suppressive protein is decreased so that immunogenicity is enhanced, and thus can be used as a pharmaceutical composition or a food composition for anti-cancer treatment.

### CROSS-REFERENCE TO RELATED APPLICATIONS

This application claims the benefit under 35 USC 119(a) of Korean Patent Application No. 10-2023-0146485, filed on Oct. 30, 2023 in the Korean Intellectual Property Office, the entire disclosure of which is incorporated herein by reference for all purposes.

### BACKGROUND OF THE INVENTION

### Field of the Invention

The present invention relates to a composition for anti-cancer treatment comprising immunogenic tumor-derived extracellular vesicles using an endoplasmic reticulum stress inducer and a method for producing the same, and more specifically relates to extracellular vesicles in which tumor cells are treated with an endoplasmic reticulum stress inducer to increase immune-activating protein and decrease immune-suppressive protein, thereby enhancing immunogenicity, and a method for producing the same.

### Description of the Related Art

Extracellular vesicles (EVs) are small-sized endoplasmic reticulum secreted by various cells and are known to play a role of cell-to-cell communication. EVs include various substances (for example, proteins, RNA, lipids, and the like) depending on the state and function of the cell and regulate information transfer between cells through the exchange of these substances.

Particularly, it has been studied that EVs secreted from tumor cells play an important role in the progression and spread of tumors. EVs derived from tumor cells are known to regulate immune responses, promote drug resistance, and support the spread of cancer cells to other tissues and organs. However, based on a deep understanding of the functions and mechanisms of these EVs, research for developing new types of cancer treatment strategies by regulating the immunogenicity of EVs is actively being conducted.

Research on the regulation of immunogenicity of extracellular vesicles can be used as a basis for not only cancer treatment using EVs, but also treatment, diagnosis, and prevention strategy development for various diseases. For example, EVs with enhanced immunogenicity can strengthen immune responses that attack cancer cells.

However, existing technologies lack methods for effectively separating and purifying extracellular vesicles with enhanced immunogenicity from tumor cells or clear methodologies for therapeutic strategies utilizing the same. In addition, the understanding of various mechanisms that regulate the immunogenicity of extracellular vesicles is still in initial stages.

Therefore, research and development on methods for producing and utilizing extracellular vesicles with enhanced immunogenicity from tumor cells are expected to contribute to innovative strategy development in the field of cancer treatment. Based on this background, the present invention proposes a technology related to extracellular vesicles with enhanced immunogenicity from tumor cells and a method for producing the same.

### SUMMARY OF THE INVENTION

The present inventors have produced extracellular vesicles obtained from tumor cells cultured in an environment which includes an endoplasmic reticulum stress inducer and have confirmed that the composition including extracellular vesicles according to the present invention can decrease immune-suppressive protein while increasing immune-activating protein compared to the existing extracellular vesicles.

As a result, an object of the present invention is to provide a composition for cancer treatment including tumor cell-derived extracellular vesicles.

Another object of the present invention is to provide a method for producing tumor cell-derived extracellular vesicles.

Still another object of the present invention is to provide a cancer treatment method using tumor cell-derived extracellular vesicles.

Still another object of the present invention is to provide a cancer treatment use of tumor cell-derived extracellular vesicles.

The present invention relates to a composition for anti-cancer treatment including immunogenic tumor-derived extracellular vesicles using an endoplasmic reticulum stress inducer and a method for producing the same. The composition according to the present invention has increased immune-activating protein and decreased immune-suppressive protein, thus the cancer treatment effect is excellent.

Hereinafter, the present invention is described in more detail.

An aspect of the present invention is a composition for producing tumor cell-derived extracellular vesicles including an endoplasmic reticulum stress inducer.

In one embodiment of the present invention, the endoplasmic reticulum stress inducer may include at least one selected from the group consisting of tunicamycin, 2-deoxy-D-glucose, brefeldin A, and thapsigargin.

The term “tunicamycin” in the present specification refers to one of the naturally occurring antibiotics, which is mainly isolated from Streptomyces and Nocardia bacteria. Tunicamycin has properties of suppressing the N-linked glycosylation process of proteins. Due to these properties, the correct folding and function of proteins within cells are hindered. Tunicamycin is widely used as a tool for protein secretion pathway research, but is toxic, and thus is not directly used in humans.

The tunicamycin can be expressed by Chemical Formula 1.

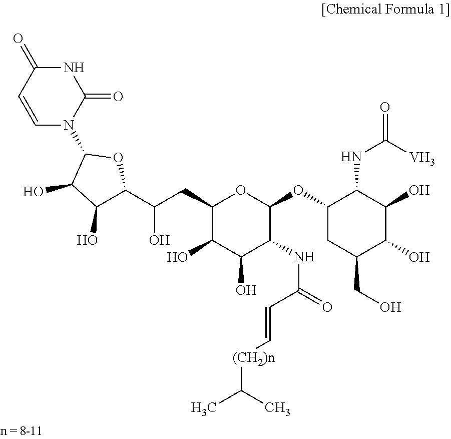

The term “2-deoxy-D-glucose” in the present specification refers to a sugar derivative in which the hydroxy group is removed from the second carbon of D-glucose and has been studied as a drug with antiviral efficacy.

The term “brefeldin A” in the present specification refers to an antibiotic compound found in nature, which is known to stop protein traffic inside cells by hindering protein transport in the Golgi body.

The term “thapsigargin” in the present specification is a terpene compound found in nature. This compound has the activity of releasing calcium from calcium storages inside cells, thereby raising the calcium concentration of cells. Thapsigargin is mainly used in the research of intracellular calcium signaling.

In one embodiment of the present invention, the composition for promoting the production of extracellular vesicles may further include a tumor cell-derived extracellular vesicle producing substance other than tunicamycin.

In one embodiment of the present invention, the composition for promoting the production of extracellular vesicles may be a composition for promoting the production of immunogenic extracellular vesicles.

The term “extracellular vesicle” in the present specification refers to a small vitreous body having various sizes and properties secreted from various cells. The extracellular vesicles act as a communication medium between cells and play an important role of transferring various types of information on the function, activity, and status of cells, and extracellular vesicles may include various biochemical substances such as protein, lipid, RNA, and DNA. In the present invention, the extracellular vesicles may be exosomes but is not limited thereto.

The term “exosomes” in the present specification refer to cell-derived endoplasmic reticulum that exist in the body fluids of almost all eukaryotic organisms and means endoplasmic reticulum having a diameter of about 30 to 300 nm, which is larger than that of LDL protein but much smaller than that of red blood cells. It is well known that exosomes can be released from cells when multivesicular bodies fuse with the cell membrane or can be released directly from the cell membrane, and perform important and specialized functions such as coagulation and intercellular signaling.

The term “tumor cells” in the present specification refer to cells that have excessive growth and division abilities that deviate from the control mechanism of normal cells. Such cells can be generated due to DNA damage or genetic mutation. Tumor cells can invade surrounding tissues or spread to other parts through blood and lymph. Uncontrolled growth of such cells leads to the formation of tumors or cancer. Not all tumor cells are malignant, and only malignant tumor cells are classified as cancer.

In the present invention, the tumor cells may include at least one selected from the group consisting of breast cancer cells, ovarian cancer cells, uterine cancer cells, prostate cancer cells, bladder cancer cells, colon cancer cells, liver cancer cells, squamous cell carcinoma cells, brain tumor cells, lymphoma cells, and myeloma cells, but are not limited thereto.

In one embodiment of the present invention, the composition according to the present invention can be used for culturing tumor cells as a medium composition and can be administered in vivo as a pharmaceutical composition for the use of in vivo extracellular vesicle production to produce immunogenic extracellular vesicles from tumor cells.

In one embodiment of the present invention, the medium composition means a composition including essential components necessary for the growth, survival and proliferation of cells in vitro and includes all culture media commonly used in the field. For example, commercially manufactured media or artificially synthesized media such as DMEM (Dulbecco's Modified Eagle's Medium), MEM (Minimal Essential Medium), BME (Basal Medium Eagle), RPMI 1640, DMEM/F-10 (Dulbecco's Modified Eagle's Medium: Nutrient Mixture F-10), DMEM/F-12 (Dulbecco's Modified Eagle's Medium: Nutrient Mixture F-12), α-MEM (α-Minimal essential Medium), G-MEM (Glasgow's Minimal Essential Medium), IMDM (Isocove's Modified Dulbecco's Medium), KnockOut DMEM, and E8 (Essential 8 Medium) can be used. However, the embodiment is not limited thereto.

In one embodiment of the present invention, the medium composition generally includes a carbon source, a nitrogen source, and a trace element component, and may further include amino acids, antibiotics, and the like.

In one embodiment of the present invention, the medium composition may be produced by adding an extracellular endoplasmic reticulum stress inducer which is a component of the present invention to a tumor cell culture medium in the related art.

In one embodiment of the present invention, the composition for promoting the production of extracellular vesicles may include tunicamycin at 0.1 μM or less, 0.09 μM or less, 0.08 μM or less, 0.07 μM or less, 0.06 μM or less, 0.05 μM or less, 0.04 μM or less, or 0.03 μM or less, and may include tunicamycin, for example, at 0.1 μM or less. However, the composition is not limited thereto.

Another aspect of the present invention is a pharmaceutical composition for treating, preventing, alleviating, or suppressing cancer including tumor cell-derived extracellular vesicles as an active ingredient.

In one embodiment of the present invention, the tumor cells may be pretreated with an endoplasmic reticulum stress inducer.

The term “endoplasmic reticulum stress inducer” in the present specification refers to a compound or a factor that causes endoplasmic reticulum stress by causing a problem in the protein folding or maturation process in the endoplasmic reticulum of a cell. The endoplasmic reticulum stress is caused due to incorrect folding, accumulation, or modification of protein, whereby the cell activates endoplasmic reticulum stress responses. The endoplasmic reticulum stress inducer according to the present invention may include a substance that causes a change in the folding and maturation of protein in an endoplasmic reticulum of a cell compared to a control group, and the endoplasmic reticulum stress inducer may be included without limitation as long as it is an endoplasmic reticulum stress inducer that is known in the art of the present invention. In the present invention, the endoplasmic reticulum stress inducer may be at least one selected from the group consisting of tunicamycin, 2-deoxy-D-glucose, brefeldin A, and thapsigargin, but is not limited thereto.

The term “pretreatment” in the present specification refers to a process of adding a specific substance to a medium of tumor cells and culturing the tumor cells. For example, pretreatment refers to a process of additionally culturing tumor cells in a medium to which tunicamycin is added.

The term “including as an active ingredient” in the present specification refers to including an amount sufficient to achieve alleviation, inhibition, prevention, or treatment activity of extracellular vesicles isolated from tumor cells or a culture thereof for a specific disease.

The term “treatment of cancer” in the present specification refers to all actions that suppress cancer, and specifically refers to an action of regulating the cycle of cancer cells or an action of inducing apoptosis of cancer cells, but is not limited thereto.

In one embodiment of the present invention, the cancer may be at least one selected from the group consisting of breast cancer, lung cancer, stomach cancer, liver cancer, blood cancer, bone cancer, pancreatic cancer, skin cancer, head or neck cancer, melanoma, uterine sarcoma, ovarian cancer, rectal cancer, anal cancer, colon cancer, fallopian tube cancer, endometrial cancer, cervical cancer, small intestine cancer, endocrine cancer, thyroid cancer, parathyroid cancer, kidney cancer, soft tissue tumor, urethral cancer, prostate cancer, bronchial cancer, glioblastoma, or bone marrow cancer, but is not limited thereto.

In one embodiment of the present invention, the cancer may be melanoma.

The pharmaceutical composition in the present invention may be administered orally and parenterally and may be administered by, for example, intravenous injection, subcutaneous injection, intramuscular injection, intraperitoneal injection, topical administration, intranasal administration, intrapulmonary administration, intrarectal administration, intrathecal administration, ocular administration, skin administration, and transdermal administration, but is not limited thereto.

The pharmaceutical composition according to the present invention may be administered in various doses depending on factors such as the formulation method, an administration method, an age, a weight, a gender, and a pathological condition of a patient, food, administration time, an administration route, an excretion rate, and response sensitivity, and may be determined or prescribed in an effective dose for the desired treatment or prevention. For example, the daily dose of the pharmaceutical composition of the present invention may be 0.0001 to 1,000 mg/kg.

According to a method that can be easily performed by a person having ordinary knowledge in the technical field to which the present invention pertains, the pharmaceutical composition according to the present invention may be produced in a unit dose form by being formulated using a pharmaceutically acceptable carrier and/or excipient, or may be produced by being introduced into a multi-dose container. Here, the formulation may be in the form of a solution, suspension, or emulsion in an oil or aqueous medium or may be in the form of extract, powder, suppository, powder, granule, tablet, or capsule, and may additionally include a dispersant or stabilizer, but is not limited thereto.

The dosage of the pharmaceutical composition according to the present invention may vary depending on an age, a weight, and a gender of a patient, a dosage form, health condition, and disease severity and may be administered once a day or several times a day divisionally at regular intervals at the discretion of a doctor or a pharmacist. For example, the daily dosage may be 1 to 1,000 μg/ml based on the content of the active ingredient. However, this is an example of an average case, and the dosage may be higher or lower depending on individual differences.

Another aspect of the present invention is a food composition for alleviating, suppressing or improving cancer including tumor cell-derived extracellular vesicles.

The food composition according to the present invention include the tumor cell-derived extracellular vesicle according to the present invention, similarly to the pharmaceutical composition described above. Therefore, the description of the common content between the two is omitted to avoid excessive complexity of the present specification.

The term “improvement” of the present invention means all actions that at least decrease a parameter related to alleviating or treating a condition, for example, the degree of symptoms.

The food composition according to the present invention may include ingredients commonly added during food production and may include, for example, proteins, carbohydrates, fats, nutrients, seasonings, and flavoring agents, but is not limited thereto.

Carbohydrates that may be included in the food composition according to the present invention may include monosaccharides such as glucose, fructose, and the like, disaccharides such as maltose, sucrose, oligosaccharides, and the like, polysaccharides such as dextrin, cyclodextrin, and the like, and sugar alcohols such as xylitol, sorbitol, erythritol, and the like, but are not limited thereto.

Flavoring agents that may be included in the food composition according to the present invention may include natural flavoring agents such as thaumatin, stevia extract, and the like, and synthetic flavoring agents such as saccharin, aspartame, and the like, but are not limited thereto.

Still another aspect of the present invention is a method for producing a tumor cell-derived extracellular vesicle, including:

a culture step of culturing tumor cells in a cell culture medium including an endoplasmic reticulum stress inducer.

In one embodiment of the present invention, a method for producing tumor cell-derived extracellular vesicles may further include:

a separation step of separating extracellular vesicles from the cultured tumor cells.

In one embodiment of the present invention, the endoplasmic reticulum stress inducer used in the culture step may include at least one selected from the group consisting of tunicamycin, 2-deoxy-D-glucose, brefeldin A, and thapsigargin.

In one embodiment of the present invention, the separation step may separate extracellular vesicles from the tumor cells by tangential flow filtration (TFF).

The culture step of the method for producing tumor cell-derived extracellular vesicles according to the present invention is a process of stimulating tumor cells by pretreating the tumor cells with the endoplasmic reticulum stress inducer. Through this process, tumor cells can produce extracellular vesicles in which immune-activating protein is increased and immune-suppressive protein is decreased compared to those in tumor cells that have not been pretreated with the above substances.

The culturing according to the present invention is a process of inducing secretion or production of extracellular vesicles from tumor cells, and the cell culture medium in the present invention may include all tumor cell culture media commonly used in the art. For example, commercially produced media or artificially synthesized media such as DMEM (Dulbecco's Modified Eagle's Medium), MEM (Minimal Essential Medium), BME (Basal Medium Eagle), RPMI 1640, DMEM/F-10 (Dulbecco's Modified Eagle's Medium: Nutrient Mixture F-10), DMEM/F-12 (Dulbecco's Modified Eagle's Medium: Nutrient Mixture F-12), α-MEM (α-Minimal essential Medium), G-MEM (Glasgow's Minimal Essential Medium), IMDM (Isocove's Modified Dulbecco's Medium), KnockOut DMEM, E8 (Essential 8 Medium), and the like may be used, but the present invention is not limited thereto.

In one embodiment of the present invention, the cell culture medium may further include components such as a carbon source, a nitrogen source, a trace element component, amino acids, antibiotics, and the like.

Still another aspect of the present invention is a method for alleviating or treating cancer including:
a step of administering, to a subject, a pharmaceutical composition including an extracellular vesicle separated from tumor cells or a culture thereof as an active ingredient.

Since the treatment method according to the present invention includes extracellular vesicles separated from a tumor cell or a culture thereof in the same manner as the composition described above, the description of the common content between the two is omitted to avoid excessive complexity of the present specification.

The term “treatment” in the present specification refers to all acts in which a disease is improved or beneficially changed by administration of the composition according to the present invention.

The term “administration” in the present specification refers to providing a predetermined substance to a patient by any appropriate method, and the route of administration of the pharmaceutical composition of the present invention may be administered orally or parenterally through all general routes as long as the pharmaceutical composition can reach the target tissue. In addition, the composition of the present invention may be administered by using any device that can transfer the active ingredient to target cells.

The term “subject” in the present specification is not particularly limited, but includes, for example, a human, a monkey, a cow, a horse, a sheep, a pig, a chicken, a turkey, a quail, a cat, a dog, a mouse, a rat, a rabbit or a guinea pig, and may be, for example, a human, but is not limited thereto.

In one embodiment of the present invention, the pharmaceutical composition according to the present invention may be administered alone but may generally be administered by being mixed with a pharmaceutical carrier selected in consideration of the administration method and standard pharmaceutical practice.

Still another aspect of the present invention is a use of a composition including tumor cell-derived extracellular vesicles as an active ingredient for alleviating, suppressing, preventing, or treating cancer.

### BRIEF DESCRIPTION OF THE DRAWINGS

FIG. 1 is a schematic diagram of producing immunogenic tumor cell-derived extracellular vesicles according to an embodiment of the present invention.

FIG. 2A is a diagram showing average particle sizes of tumor cell-derived extracellular vesicles (bTDEs) that are not treated with any specific substance according to an embodiment of the present invention.

FIG. 2B is a diagram showing average particle sizes of tumor cell-derived extracellular vesicles (mTDEs) that are treated with metformin according to an embodiment of the present invention.

FIG. 2C is a diagram showing average particle sizes of tumor cell-derived extracellular vesicles (iTDEs) that are treated with tunicamycin according to an embodiment of the present invention.

FIG. 3A is a result obtained by evaluating viability of tumor-derived cells when treated with tunicamycin at different concentrations according to an embodiment of the present invention.

FIG. 3B is a result obtained by evaluating viability of tumor-derived cells when treated with metformin at different concentrations according to an embodiment of the present invention.

FIG. 4A is a diagram showing changes in expression patterns of immune-activating protein (HMGB1, HSP70) in tumor cell-derived extracellular vesicles (bTDEs) that are not treated with any specific substance, tumor cell-derived extracellular vesicles (mTDEs) that are treated with metformin, and tumor cell-derived extracellular vesicles (iTDEs) that are treated with tunicamycin according to an embodiment of the present invention (A. expression patterns of immune-activating protein

FIG. 4B is a diagram showing changes in expression patterns of expression patterns of immune-suppressive protein (CD47, S100A9) in tumor cell-derived extracellular vesicles (bTDEs) that are not treated with any specific substance, tumor cell-derived extracellular vesicles (mTDEs) that are treated with metformin, and tumor cell-derived extracellular vesicles (iTDEs) that are treated with tunicamycin according to an embodiment of the present invention.

FIG. 5A is a graph showing results obtained by observing changes in the expression of CD80 by treating dendritic cells with tumor cell-derived extracellular vesicles (bTDEs) that are not treated with any specific substance, tumor cell-derived extracellular vesicles (mTDEs) that are treated with metformin, tumor cell-derived extracellular vesicles (iTDEs) that are treated with tunicamycin, and LPS according to an embodiment of the present invention.

FIG. 5B is a graph showing results obtained by observing changes in the expression of CD86 by treating dendritic cells with tumor cell-derived extracellular vesicles (bTDEs) that are not treated with any specific substance, tumor cell-derived extracellular vesicles (mTDEs) that are treated with metformin, tumor cell-derived extracellular vesicles (iTDEs) that are treated with tunicamycin, and LPS according to an embodiment of the present invention.

FIG. 5C is a graph showing results obtained by observing changes in the expression of CCR7 by treating dendritic cells with tumor cell-derived extracellular vesicles (bTDEs) that are not treated with any specific substance, tumor cell-derived extracellular vesicles (mTDEs) that are treated with metformin, tumor cell-derived extracellular vesicles (iTDEs) that are treated with tunicamycin, and LPS according to an embodiment of the present invention.

FIG. 6A is a drawing showing volumes of tumors on day 21 of treatment, of B16F10 melanoma-bearing mice, after being treated with tumor cell-derived extracellular vesicles (bTDEs) that are not treated with any specific substance, tumor cell-derived extracellular vesicles (mTDEs) that are treated with metformin, and tumor cell-derived extracellular vesicles (iTDEs) that are treated with tunicamycin according to an embodiment of the present invention.

FIG. 6B is a picture showing volumes of tumors on day 21 of treatment, of B16F10 melanoma-bearing mice, after being treated with tumor cell-derived extracellular vesicles (bTDEs) that are not treated with any specific substance, tumor cell-derived extracellular vesicles (mTDEs) that are treated with metformin, and tumor cell-derived extracellular vesicles (iTDEs) that are treated with tunicamycin according to an embodiment of the present invention.

FIG. 6C is a drawing showing weight of tumors on day 21 of treatment, of B16F10 melanoma-bearing mice, after being treated with tumor cell-derived extracellular vesicles (bTDEs) that are not treated with any specific substance, tumor cell-derived extracellular vesicles (mTDEs) that are treated with metformin, and tumor cell-derived extracellular vesicles (iTDEs) that are treated with tunicamycin according to an embodiment of the present invention.

FIGS. 7A-7D are drawings showing results obtained by analyzing immune cells in the bodies by administering, to mouse models, tumor cell-derived extracellular vesicles (bTDEs) that are not treated with any specific substance, tumor cell-derived extracellular vesicles (mTDEs) that are treated with metformin, and tumor cell-derived extracellular vesicles (iTDEs) that are treated with tunicamycin according to an embodiment of the present invention (FIG. 7A. the ratio of activated dendritic cells in lymph nodes derived from each mouse, FIG. 7B. the ratio of cytotoxic T lymphocytes distributed in tumors of each mouse, and FIGS. 7C and 7D. the antigen reactivity evaluation in spleen cells derived from each mouse).

FIG. 7A is a diagram showing the ratio of activated dendritic cells in lymph nodes derived from mice after administering tumor cell-derived extracellular vesicles (bTDEs) that are not treated with any specific substance, tumor cell-derived extracellular vesicles (mTDEs) that are treated with metformin, and tumor cell-derived extracellular vesicles (iTDEs) to a mouse model according to an embodiment of the present invention.

FIG. 7B is a diagram showing the ratio of cytotoxic T lymphocytes distributed in the tumors of mice after administering tumor cell-derived extracellular vesicles (bTDEs) that are not treated with any specific substance, tumor cell-derived extracellular vesicles (mTDEs) that are treated with metformin, and tumor cell-derived extracellular vesicles (iTDEs) to a mouse model according to an embodiment of the present invention.

FIG. 7C is a diagram evaluating the reactivity to the Ki-67 antigen in splenocytes after administering tumor cell-derived extracellular vesicles (bTDEs) that are not treated with any specific substance, tumor cell-derived extracellular vesicles (mTDEs) that are treated with metformin, and tumor cell-derived extracellular vesicles (iTDEs) to a mouse model according to an embodiment of the present invention.

FIG. 7D is a diagram evaluating the reactivity to the Grz-B antigen in splenocytes after administering tumor cell-derived extracellular vesicles (bTDEs) that are not treated with any specific substance, tumor cell-derived extracellular vesicles (mTDEs) that are treated with metformin, and tumor cell-derived extracellular vesicles (iTDEs) to a mouse model according to an embodiment of the present invention.

FIG. 8A is a diagram showing the tumor volume in each treatment group of B16F10 melanoma-bearing mice, after partial resection of the tumor, following the administration of tumor cell-derived extracellular vesicles (bTDEs) that are not treated with any specific substance, tumor cell-derived extracellular vesicles (mTDEs) that are treated with metformin, and tumor cell-derived extracellular vesicles (iTDEs) to a mouse model according to an embodiment of the present invention.

FIG. 8B is a diagram showing the survival rates of B16F10 melanoma-bearing mice, after partial resection of the tumor, following the administration of tumor cell-derived extracellular vesicles (bTDEs) that are not treated with any specific substance, tumor cell-derived extracellular vesicles (mTDEs) that are treated with metformin, and tumor cell-derived extracellular vesicles (iTDEs) to a mouse model according to an embodiment of the present invention.

### DETAILED DESCRIPTION OF THE PREFERRED EMBODIMENTS

Hereinafter, the present invention is described in more detail with reference to the following examples. However, these examples are only for illustrating the present invention, and the scope of the present invention is not limited by these examples.

### Example 1: Separation of Tumor Cell-Derived Extracellular Vesicles by Tunicamycin Treatment

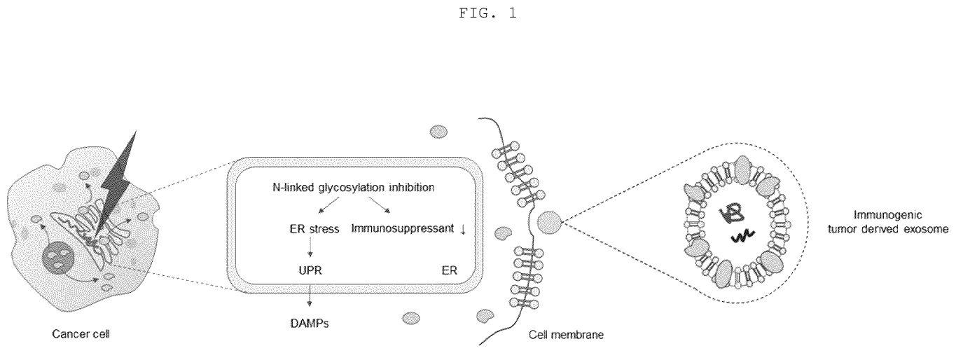

In order to compare the characteristics of extracellular vesicles by tunicamycin treatment on tumor cells, tumor endoplasmic reticulum was treated with metformin separately from tunicamycin to obtain extracellular vesicles as shown in FIG. 1.

For extracellular vesicles (bTDE) that are not treated with any specific substance, B16F10 cells, a melanoma cell line, were rinsed twice with PBS and cultured in a serum-free DMEM medium for 24 hours, and then the cell culture supernatant was collected, centrifuged (2,500 g, 20 minutes), and filtered through a 0.22 μm filter to remove cell debris and large vesicles. Afterwards, the extracellular vesicles were separated from the in vitro cell culture medium using a tangential flow filtration (TFF) system with an Omega membrane filter capsule (Pall Corporation, Port Washington, NY) with a molecular weight cutoff of 300 kDa.

In case of the separation of the extracellular vesicles treated with tunicamycin or metformin (iTDE and mTDE), B16F10 cells which are the cell line were rinsed twice with PBS, cultured for 24 hours in a serum-free DMEM medium including 0.05 μM of tunicamycin or 5 mM of metformin, and then separated from the in vitro cell culture medium using a tangential flow filtration system in the same manner as described above.

Each extracellular vesicle (PBS containing 2×10⁹ particles/ml) was analyzed by a nanoparticle tracking analysis system (NTA; Nano Sight LM10, Malvern Instruments, UK). The time-resolved Brownian motion of the particles was measured three times for 30 seconds, and the size distribution of the particles was measured through this.

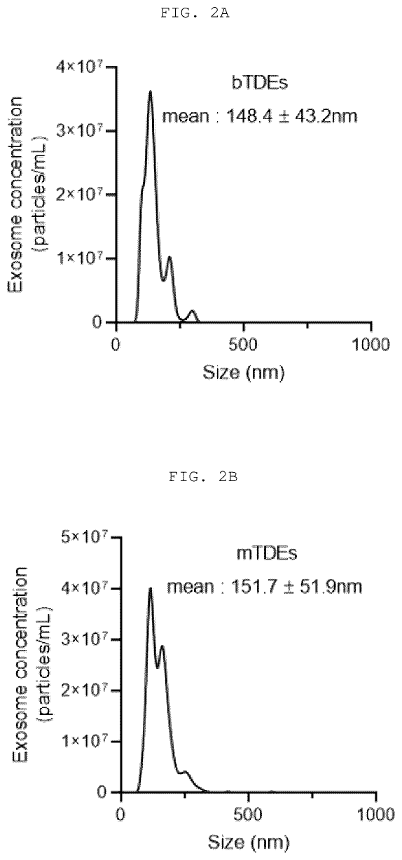

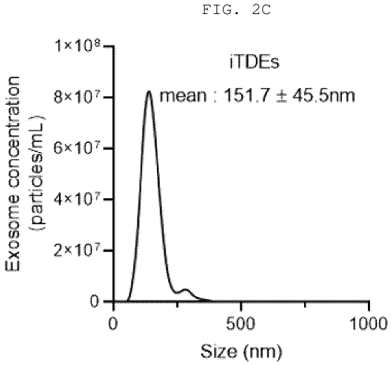

As a result of the experiment, as can be seen in FIG. 2A to 2C, the relative number of extracellular vesicles in the culture medium according to the treatment with tunicamycin and metformin was measured using a nano particle tracking analyzer, and it was confirmed that the size of the extracellular vesicle particles did not change depending on the drug treatment. This result indicates that the drug treatment does not affect the physicochemical properties of the extracellular vesicles.

### Example 2: Viability Evaluation of Tumor Cells According to Tunicamycin Treatment

Cell viability was evaluated after treatment with tunicamycin and metformin at different concentrations.

To conduct the cytotoxicity evaluation of each pretreatment substance (tunicamycin and metformin) on the melanoma cell line B16F10, 3-(4,5-dimethylthiazol-2-yl)-2,5-diphenyltetrzolium bromide (MTT) assay was performed. First, 10⁴ cells were seeded in each 96-well plate and cultured for 24 hours. Then, cells in each well were treated with each substance at a given concentration and cultured for an additional 24 hours. After the supernatant was discarded, the cells were treated with an MTT-containing medium at the concentration of 0.5 mg/ml and left for two hours, and the intracellular formazan crystals were dissolved in each well using dimethylsulfoxide. Then, the cytotoxicity was analyzed by measuring the absorbance at 570 nm by using a microplate reader.

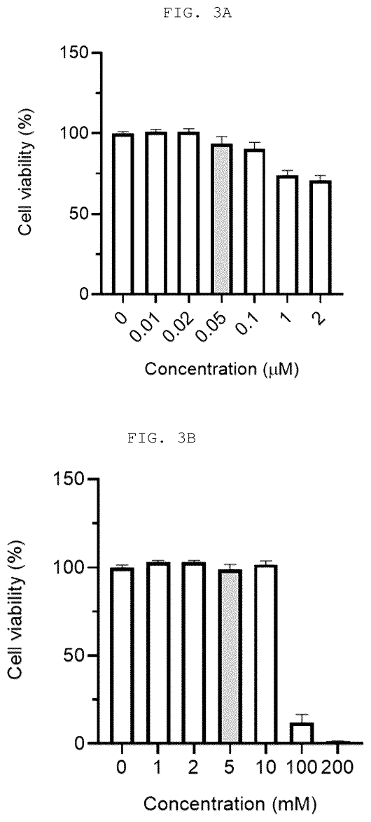

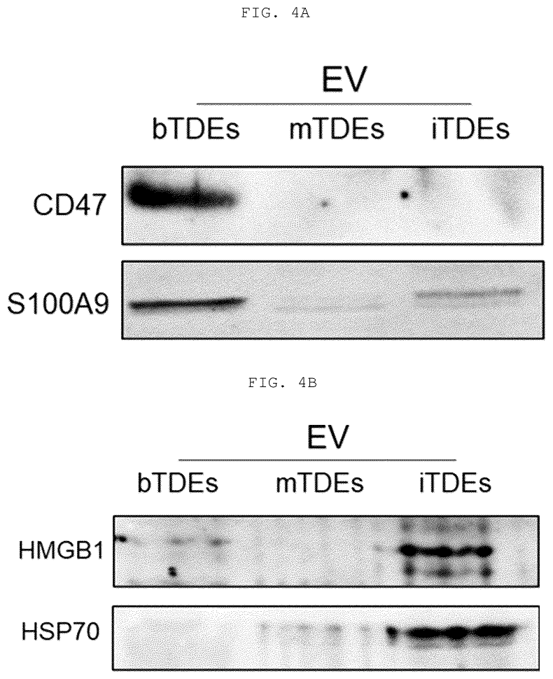

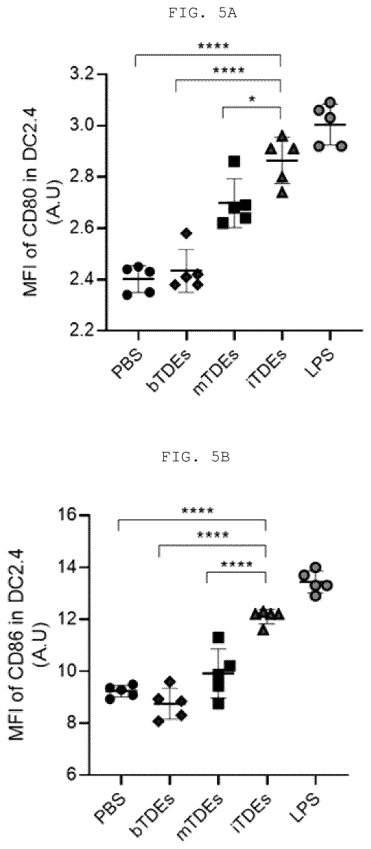

As a result of the experiment, as can be seen in FIG. 3A to 3B, there was no significant decrease in the viability of tumor cells at concentrations of 0.05 μM or less of tunicamycin, but a decrease in cell viability due to apoptosis was observed at concentrations of 0.1 μM or more. When apoptosis occurs, apoptotic bodies may be mixed with the extracellular vesicles in the culture medium. Therefore, according to the results described above, the concentration of tunicamycin was determined as the condition of 0.05 μM, the highest concentration at which an increase in immune-activating proteins (DAMPs) and a decrease in immune-suppressive proteins were induced while no decrease in cell viability was observed. Similarly, the concentration of metformin was determined as the condition of 5 mM, the highest concentration at which a decrease in immune-suppressive proteins was induced and no decrease in cell viability was observed.

### Example 3: Protein Analysis of Extracellular Vesicles Through Western Blotting

Each extracellular vesicle was dispersed in Radio-Immunoprecipitation Assay buffer, and membrane lysis was performed using a probe type sonicator. Afterwards, the samples were separated by size through 12% SDS (sodium dodecyl sulfate) polyacrylamide gel electrophoresis and blotted onto a polyvinylidene difluoride membrane (Millipore, Burlington, MA, USA). Staining was performed overnight at 4° C. using anti-HMGB1 (ab18256, Abcam, Cambridge, UK, 1:1000), anti-HSP70 (ab2787, Abcam, Cambridge, UK, 1:1000), anti-CD47 (ab214453, Abcam, Cambridge, UK, 1:1000), and anti-S100A9 (PA146489, Invitrogen, Waltham, MA, 1:1000) primary antibodies. After rinsing with a Tris buffer containing 0.1% Tween, staining was performed for one hour at room temperature using horseradish peroxidase-conjugated secondary antibodies, and chemiluminescence of the secondary antibody-stained area was imaged using a chemiluminescent substrate solution.

As a result of confirming changes in immune-activating protein and immune-suppressive protein of tumor cell-derived extracellular vesicles (iTDE) treated with 0.05 μM of tunicamycin and tumor cell-derived extracellular vesicles (mTDE) treated with 5 mM of metformin by western blotting, compared to normal tumor-derived extracellular vesicles (bTDE), iTDE showed an increase in HMGB1 and HSP70, immune-activating proteins, and a decrease in CD47 and S100A9, immune-suppressive proteins, whereas mTDE showed only a decrease in CD47 and S100A9, immune-suppressive proteins.

### Example 4: Evaluation of Dendritic Cell Activation Ability

In order to confirm the effect of iTDE on dendritic cells according to an example, cell lines of dendritic cells, DC2.4 cell lines, were treated with each of bTDE, mTDE, and iTDE in the culture medium.

The mouse dendritic cell lines, DC2.4 cell lines, were seeded in 96 wells by 10⁴ cells each. After one day of incubation, 10⁷ cells of each extracellular vesicle per well were treated, and LPS treatment was performed using a medium containing 100 ng/ml of LPS. After 48 hours of culture, the cell suspension rinsed three times with PBS was stained at 4° C. for 30 minutes using anti-mouse FITC-CD80 (104705, Biolegend, San Diego, CA), anti-mouse PE/Dazzle 594-CD86 (105041, Bio legend, San Diego, CA), and anti-mouse PE/Dazzle 594-CCR7 (120121, Biolegend, San Diego, CA). The stained samples were rinsed three times with PBS and analyzed using a flow cytometer (MACSQuant VYB, MiltenyiBiotec).

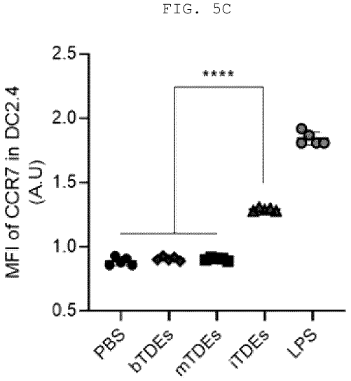

As a result of the experiment, as can be seen in FIG. 5A to 5C, it was observed that the expression levels of CD80, CD86, and CCR7, activation marker protein of dendritic cells, were significantly increased in the iTDE treatment group compared to other treatment groups. This confirmed that iTDE can effectively activate dendritic cells.

### Example 5: Evaluation of Anti-Cancer Effect in Melanoma Mouse Model

The anti-cancer effect of iTDE was evaluated on a B16F10-bearing animal model, a mouse melanoma cell line.

To prepare tumor-bearing mice, 1×10⁶ melanoma B16F10 cells were suspended in 100 μl of PBS and inoculated subcutaneously into the right flank of C57BL/6 mice. On day 10 after inoculation, B16F10 tumor-bearing mice were randomly divided into four treatment groups (n=5 mice per group): saline, bTDEs, mTDEs, and iTDEs. Mice were injected subcutaneously with 10⁹ extracellular vesicle suspensions in a volume of 100 μl. Tumor volumes and body weights were recorded daily and calculated as follows: volume (mm³)=0.5×length (mm)×width (mm)×width (mm). On day 21, mice were euthanized, and tumor tissues, related organs, and blood samples were collected for further analysis.

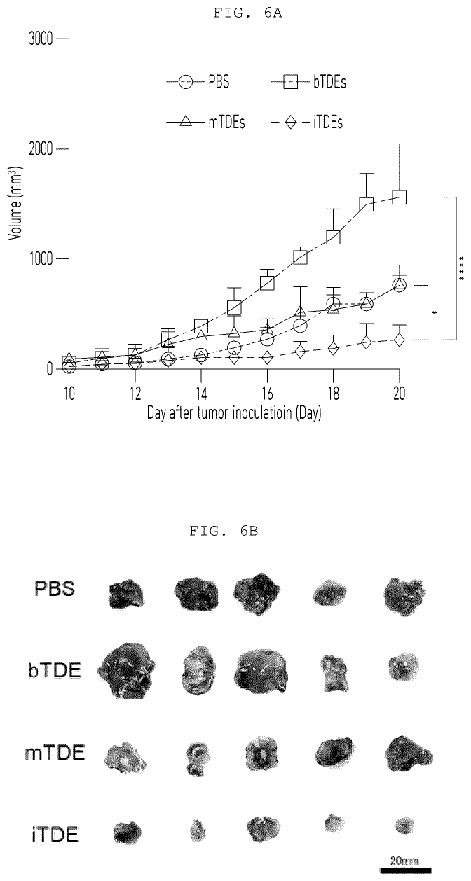

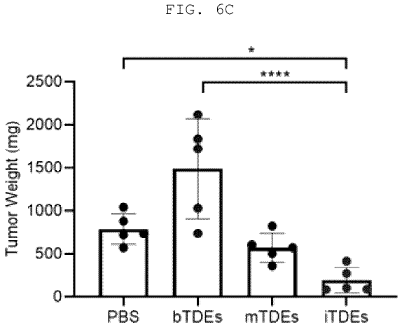

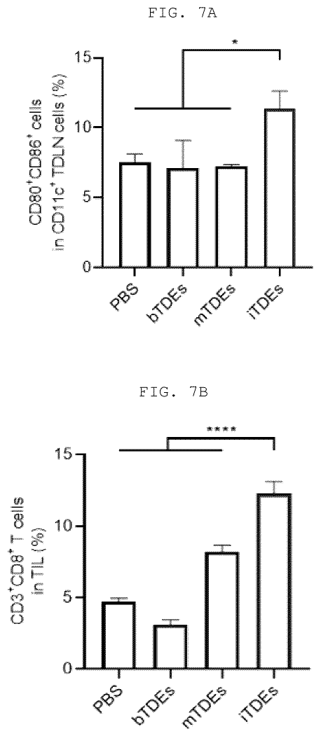

As a result of the experiment, as can be seen in FIG. 6A to 6C, bTDE promoted tumor growth, whereas mTDE suppressed the expression of immune-suppressive proteins and did not affect tumor growth. Meanwhile, iTDE showed an effect of suppressing tumor growth, unlike other extracellular vesicles (see FIGS. 6A and 6B), which was consistent with photographs and weight results of tumors obtained from animal models on day 21 after tumor transplantation (see FIG. 6C).

### Example 6: Analysis of Ratio of Immune Cells in Melanoma Mouse Model

Ratios of immune cells in the body were compared and analyzed in the animal models injected with iTDE.

After sacrificing the animal models on day 21, lymph nodes surrounding the tumor were separated from the groin area of the right leg of the animal. The separated lymph nodes were mechanically dissociated and sequentially passed through 70 and 40 μm cell strainers to obtain a single cell suspension, and then RBC lysis was performed. Afterwards, 2×10⁶ sorted cells were blocked with anti-mouse CD16/32 antibody on ice for 15 minutes, and fluorescent staining was performed for 30 minutes at 4° C. in the dark using the following antibodies: anti-FITC-CD80 and anti-CD86-PE/Dazzle™ 594. The stained samples were rinsed three times with PBS and analyzed using a flow cytometer (MACSQuant VYB, MiltenyiBiotec).

Dissociation of the harvested B16F10 tumors was performed using a mouse tumor dissociation kit (Miltenyi Biotec) and a Gentle MACS dissociator (Miltenyi Biotec) according to the manufacturer's instructions. The tumor homogenate was sequentially passed through 70 and 40 μm cell strainers to obtain a single cell suspension, and then RBC lysis was performed. The above cells were further magnetically sorted using Miltenyi CD45 TIL microbeads to analyze the intratumoral T cell and myeloid cell populations, respectively. 2×10⁶ sorted cells were then blocked with anti-mouse CD16/32 antibody on ice for 15 minutes, and fluorescent staining was performed for 30 minutes at 4° C. in the dark using the following antibodies: anti-CD3-FITC (clone 145-2C11, BioLegend) and anti-CD8-PE/Dazzle™ 594 (clone 53-6.7, BioLegend). The stained samples were rinsed three times with PBS and analyzed using a flow cytometer (MACSQuant VYB, MiltenyiBiotec).

To analyze the proliferation and effector function of splenic T cells, spleen cells were physically dissociated and seeded in 24-well plates at a density of 1×10⁶ cells. Cells were stimulated in the presence of 100 IU/ml of IL-2 and tumor lysate for 48 hours. After staining with anti-PE/Dazzle 594-Ki-67 and anti-FITC-Grn-b, the stained samples were rinsed three times with PBS and analyzed using a flow cytometer (MACSQuant VYB, MiltenyiBiotec).

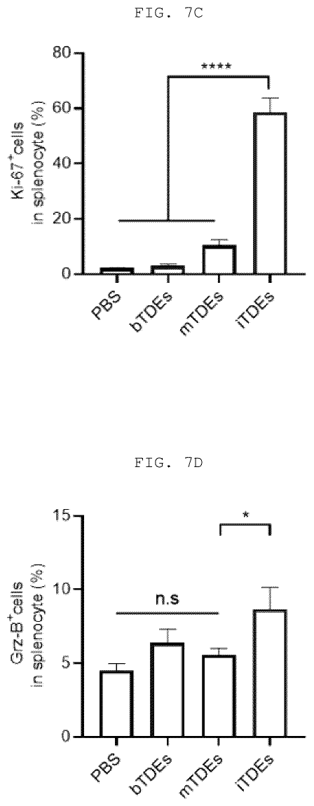

As a result of the experiment, as can be seen in FIG. 7A to 7D, the proportion of activated dendritic cells in the lymph nodes surrounding the tumor was the largest in the iTDE treatment group (see FIG. 7A), and the proportion of activated cytotoxic T lymphocytes was also the largest in the iTDE treatment group (see FIG. 7B). This indicates that the activated dendritic cells effectively induced the activation and division of cytotoxic T lymphocytes that attack the tumor. As a result of evaluating the antigen reactivity of spleen cells of the above animal model, it was observed that the expression level of Ki-67, a T lymphocyte proliferation marker protein, and the expression level of Granzyme B, a cytotoxicity marker protein, were significantly increased in the iTDE treatment group compared to other treatment groups (see FIG. 7C and FIG. 7D). These results suggest that immune cells that have encountered an antigen once can induce a stronger immune response when the immune cells encounter the same antigen again.

### Example 7: Tumor Recurrence Evaluation

Since iTDE can be used by being separated from a surgically removed tumor, a tumor recurrence model was formed through surgery in an animal model bearing B16F10, a mouse melanoma cell line, and then a tumor recurrence evaluation was performed.

To prepare tumor-bearing mice, 1×10⁶ melanoma B16F10 cells were suspended in 100 μl of PBS and subcutaneously inoculated into the right flank of C57BL/6 mice. On day 12 after inoculation, the tumor was cut into the volume corresponding to a half of the cross-sectional area, sutured with surgical thread, and disinfected to form a tumor recurrence model, which was divided into four treatment groups (n=6 mice per group): saline, bTDEs, mTDEs, and iTDEs. From day 13 to day 19 after tumor injection, 10⁹ extracellular vesicle suspensions in a volume of 100 μl were subcutaneously injected. The tumor volumes and body weights were recorded daily and calculated as follows: volume (mm³)=0.5×length (mm)×width (mm)×width (mm). From day 20 to day 25, the viability evaluation of the animals was performed.

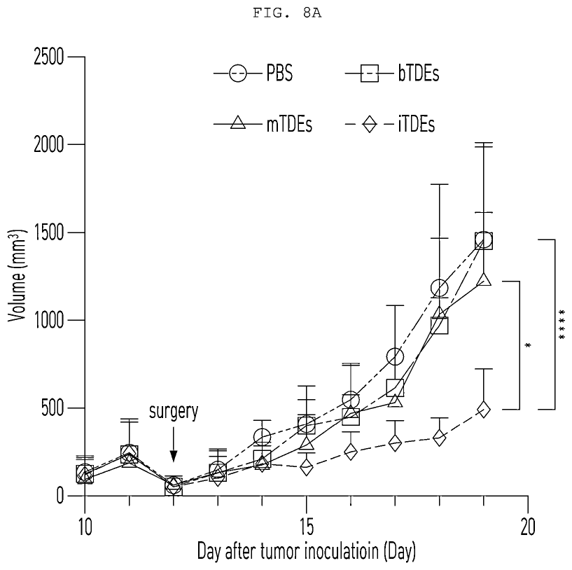

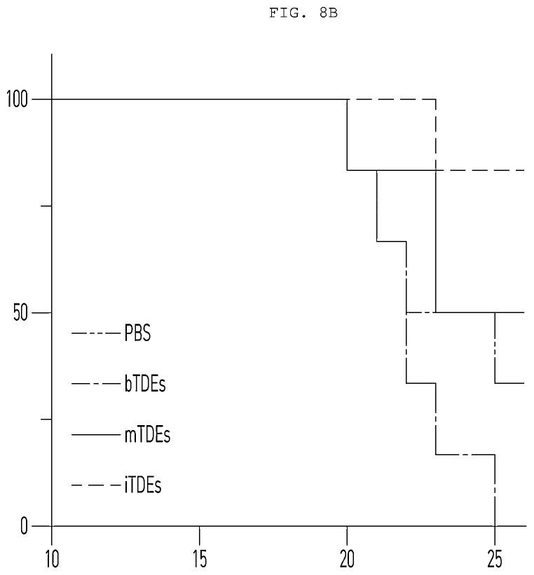

As a result of the experiment, as can be seen in FIG. 8A to 8B, bTDE and mTDE rapidly promoted tumor regrowth, but iTDE showed an effect of suppressing tumor regrowth (see FIG. 8A). In addition, the viability was the highest in the above surgical animal model injected with iTDE (see FIG. 8B). The above results indicate that the iTDE proposed in the present invention is expected to improve the overall prognosis of patients undergoing surgery by suppressing tumor recurrence through increasing the expression of immune-activating proteins and decreasing the expression of immune-suppressing proteins.

The present invention relates to a composition for anti-cancer treatment including immunogenic tumor-derived extracellular vesicles using an endoplasmic reticulum stress inducer and a method for producing the same, and the composition according to the present invention includes extracellular vesicles in which tumor immune-activating protein is increased and immune-suppressive protein is decreased so that immunogenicity is enhanced, and thus can be used as a pharmaceutical composition or a food composition for anti-cancer treatment.

What is claimed is:

1. A method for producing a tumor cell-derived extracellular vesicle, comprising step of: contacting tumor cells with an endoplasmic reticulum stress inducer.

2. The method of claim 1, wherein the endoplasmic reticulum stress inducer includes at least one selected from the group consisting of tunicamycin, 2-deoxy-D-glucose, brefeldin A, and thapsigargin.

3. The method of claim 1, wherein the tumor cell includes at least one selected from the group consisting of breast cancer cells, ovarian cancer cells, uterine cancer cells, prostate cancer cells, bladder cancer cells, colon cancer cells, liver cancer cells, squamous cell carcinoma cells, brain tumor cells, lymphoma cells, and myeloma cells.

4. A method for treating cancer comprising step of: administering, to a subject, a tumor cell-derived extracellular vesicle as an active ingredient.

5. The method of claim 4, wherein the tumor cell is pretreated with an endoplasmic reticulum stress inducer.

6. The method of claim 5, wherein the endoplasmic reticulum stress inducer includes at least one selected from the group consisting of tunicamycin, 2-deoxy-D-glucose, brefeldin A, and thapsigargin.

7. The method of claim 4, wherein the tumor cell includes at least one selected from the group consisting of breast cancer cells, ovarian cancer cells, uterine cancer cells, prostate cancer cells, bladder cancer cells, colon cancer cells, liver cancer cells, squamous cell carcinoma cells, brain tumor cells, lymphoma cells, and myeloma cells.

8. The method of claim 4, wherein the cancer is at least one selected from the group consisting of breast cancer, lung cancer, stomach cancer, liver cancer, blood cancer, bone cancer, pancreatic cancer, skin cancer, head or neck cancer, melanoma, uterine sarcoma, ovarian cancer, rectal cancer, anal cancer, colon cancer, fallopian tube cancer, endometrial cancer, cervical cancer, small intestine cancer, endocrine cancer, thyroid cancer, parathyroid cancer, kidney cancer, soft tissue tumor, urethral cancer, prostate cancer, bronchial cancer, glioblastoma, or bone marrow cancer.

9. The method of claim 5, wherein, in the extracellular vesicles, immune-activating protein is increased compared to those in tumor cell-derived extracellular vesicles that are not pretreated with the endoplasmic reticulum stress inducer.

10. The method of claim 5, wherein, in the extracellular vesicles, immune-suppressive protein is decreased compared to those in tumor cell-derived extracellular vesicles that are not pretreated with the endoplasmic reticulum stress inducer.
